(19) United States
(12) Patent Application Publication
(10) Pub. No.: US 2026/0249035 A1
(43) Pub. Date: Aug. 27, 2026

(54) DEVICE FOR REGULATING OXYGEN FOR AUTOMATED OXYGEN THERAPY

(71) Applicant: O2MATIC APS, Herlev (DK)

(72) Inventors: Ejvind Frausing, Herlev (DK); Farzad Saber, Herlev (DK); Görgen Okan Ilker, Herlev (DK)

(21) Appl. No.: 18/877,331

(22) PCT Filed: Jun. 30, 2023
(86) PCT No.: PCT/EP2023/068129

§ 371 Date: Dec. 20, 2024

(30) Foreign Application Priority Data
Jun. 30, 2022 (EP) 22182386.7

(51) Int. Cl. A61M 16/20; A61M 16/00

(52) U.S. Cl. CPC A61M 16/203; A61M 2016/0033; A61M 2202/0208; A61M 2205/18; A61M 2205/3303; A61M 2205/3334; A61M 2205/52; A61M 2230/205

(57) ABSTRACT

According to the invention there is herein detailed a method for automated oxygen therapy and a double closed-loop regulated device for regulating oxygen for automated oxygen therapy, the device and method comprising use of a controller configured for controlling the provided flow of oxygen through the valve by adjusting the valve in response to feedback from a flowmeter and a sensor; the controller further configured for executing a control algorithm in response to measured physiological data received from the sensor indicating a patient activity involving physical exercise; the control algorithm permitting an iterative adaptation of the flow of oxygen through the valve using a predictive oxygen consumption model for compensating for a heightened patient oxygen consumption during physical exercise.

### TECHNICAL FIELD

With the present invention there is detailed a device for regulating oxygen for automated oxygen therapy, wherein the device can provide delivery of oxygen to a patient adaptive to a change in the condition of the patient and to a physical activity performed by the patient.

### BACKGROUND

The control of oxygen dosing for patients admitted to hospital with hypoxemic respiratory failure has virtually been unchanged for 100 years since Haldane in 1917 described the dosing of oxygen from an oxygen tank with a manual flowmeter, a reservoir bag and a hose to a mask over the patients nose and mouth. Improvements due to the introduction of the pulse oximeter has since provided a non-invasive possibility of measuring the saturation of oxygen in the blood (SpO₂), however, control of oxygen flow is mostly done manually by the nursing staff after intermittent readings of patient SpO₂.

Depending on how critically ill the patient is, manual control of the oxygen flow is done from 2 to 48 times a day (or more) and is thus a very time-consuming task. Furthermore, the intermittent and manual prescribing and administration of oxygen is often not in adherence with treatment guidelines, which for many diseases prescribe adjustment to a patient oxygen SpO₂-regimen without occurrence of hypoxemia (low SpO₂) nor hyperoxia (high SpO₂). Particularly, when manual SpO₂ control is too infrequent, repeated hypoxemia and/or hyperoxia can occur.

In Chronic Obstructive Pulmonary Disease (COPD) both hypoxemia and hyperoxia have been shown to increase mortality during hospitalization and to increase the risk of serious adverse events, such as respiratory failure with CO₂-retention in the blood and need for mechanical ventilation.

In the current market, few devices exist for automated oxygen delivery. Present Applicant markets a device for regulating oxygen in automated oxygen therapy, O2matic, based on the technology detailed in WO 2006/110812 for continuous patient use in hospitals and care stations.

In WO 2006/110812 (cf. FIG. 1) there is detailed a device (1) and method for limiting adverse events during supplemental oxygen therapy, wherein the oxygen flow between a patient (4) and an oxygen source (2) is controlled with an adjustable valve (11), such as a proportional solenoid capable of constraining flowrates within a continuous range. The flowrate of oxygen is accurately controlled in a double closed-loop regulation comprising flowrate measurements (12) and continuous measurements (3) of vital patient's physiological data for automatically establishing an optimum therapeutic oxygen flowrate. During use, controller signal filtering (14) is commonly provided for improving the overall response and stability, wherein the control algorithm varies flowrates to minimize disturbances in the patient feedback measurements and the double-loop feedback allows the system to settle iteratively on a stable value for the oxygen supply to the patient.

In an aspect of the device manufactured by present Applicant (c.f. WO 2021/005168), the marketed device using double closed-loop oxygen regulation is suitable for adaptively regulating oxygen for automated oxygen therapy without feedback from the sensor based on a prior established prediction, established during a first and second prediction time period, and executed during a third execution time period, the sum of the first and second time periods being smaller, usually significantly smaller than the third time period, whereby treatment compliance is improved in home care patients otherwise forced to wear a sensor continuously for adequate automated oxygen therapy.

In recent research, however, using automated oxygen therapy during physical exercise in the relevant patient group has been investigated, c.f., e.g., Vivodtzev et al. Thorax 2018, v74, pp. 298-301, or in Kofod et al., J. Clin. Med. 2021, v10, pp. 4820-4831, the present inventor contributing. In these studies, it was realized that patients' oxygen levels closed loop regulated automated oxygen therapy devices, including in the study of Kofod et al. the O2matic device of present Applicant, were improved during physical exercise (walking) if the oxygen received was adjusted automatically during the physical exercise (Observed as longer walking distance, improved physical endurance and walking speed, while at the same time halving the degree of dyspnea experienced by the patient). In the latter double-blinded study by Kofod, a near doubling was observed from about 5.5 minutes of walking capability in the control group receiving constant oxygen supply to about 10.9 minutes of walking capability in the treatment group receiving a supply of oxygen regulated in response to a given patient's measured physiological data comprising SpO₂ and pulse rate from a pulse oximeter during exercise.

However, the present inventor has realized that the studies performed in the prior art have been limited by the reliance on the measurement of patient SpO₂ and pulse rate from the pulse oximeter during exercise testing, (for providing regulation feedback to the controller (13) for adjusting the oxygen flow and, consequently, the oxygen supplied to the patient), as the measured physiological data of the patient (4), by their nature are lagging behind the oxygen needed at any time, in particularly during rapid metabolism stages such as during exercise. Consequently, regulation of the oxygen flow received from a device for automated oxygen therapy is reactive rather than predictive in nature during physical exertion.

For overcoming this problem in automated oxygen therapy, the present inventor has developed, and herein proposes, the use of a predictive regulation algorithm (100) for automated oxygen therapy, in particular for automated oxygen therapy during a patient's physical exertion, the predictive regulation algorithm providing improved clinical therapy outcome to a patient receiving automated oxygen therapy, in particular a patient receiving automated oxygen therapy during physical exertion. Using the herein presented algorithms, regulation lag-time introduced by the inherent reactive reliance on the pulse oximeter sensor providing physiological data from the patient to the automated oxygen therapy device can be improved upon.

### ABBREVIATIONS

SpO₂: Oxygen-saturation measured by pulse oximetry.
SpO₂ interval: The limits for acceptable SpO₂ as set in the present device.
SpO₂ target: The middle SpO₂ value in the SpO₂ interval. E.g., if interval is set to 88-92% SpO₂, target will be 90%.
SpO₂ actual: The actual measured SpO₂ averaged over the last 15 seconds.
Flow: The oxygen flow from present device delivered to the patient.
FiO₂: Fraction of O₂ in inspired air-derivable from Table 1 describing relation between flow and FiO₂.
PaO₂: Partial pressure of oxygen in arterial blood-derived from Table 2 describing the relation between SpO₂ and PaO₂.
PAO₂: Partial pressure of oxygen in the alveolar units in the lung. Calculated from the gas equation (Table 3).
PAO₂-PaO₂: Alveolar-arterial oxygen-difference, which is a measure of the pulmonary condition. The higher the value, the worse the condition.
FR: Flow-response. A multiplication factor allowing for rapid changes in oxygen-flow.
P_{ATM}: Atmospheric pressure in kPa.
P_{H2O}: Pressure of water in alveolar air in kPa.
PaCO₂: Pressure of carbon dioxide in arterial blood.
RER: Respiratory exchange ratio. The ratio between produced carbon dioxide and consumed oxygen.

### SUMMARY OF THE INVENTION

According to the present invention there is herein disclosed a double closed-loop regulated device (10) for regulating a flow of oxygen for automated oxygen therapy, the device (10) comprising an oxygen flow path (5a-c) for passing a flow of oxygen from a source of oxygen (2) via the device (10) for providing a controlled flow of oxygen, the device (10) comprising an adjustable valve (11) and a flowmeter (12) arranged consecutively on the oxygen flow path (5a-c), and a controller (13) configured (15) for controlling a provided flow of oxygen through the adjustable valve (11) by adjusting the adjustable valve (11) in response to feedback (16,31) from the flowmeter (12) and a sensor (3) for measuring physiological data comprising patient SpO₂ and pulse rate, the sensor (3) configured for being operatively connected (31) to the controller (13); the controller (13) further configured for executing a control algorithm (100) in response to a change in measured physiological data received from the sensor (3) indicating a patient activity involving physical exertion; the control algorithm (100) permitting an iterative adaptation of the flow of oxygen through the adjustable valve (11) by using a predictive oxygen consumption model (200) for compensating for an increased patient oxygen consumption during physical exertion.

In an embodiment thereof, the predictive oxygen consumption model (200) is given by Equation (1b).

$$Δ Flow = k \frac{{SpO}_{2} ( T ) - {SpO}_{2} ( A )}{Δ {Fi O}_{2} ( N - A )} \qquad ( 1 b )$$

### BRIEF DESCRIPTION OF THE DRAWINGS

FIG. 1: Prior art device for automated oxygen therapy

FIG. 2: Device for automated oxygen therapy of the invention

FIG. 3: Flowchart for automated oxygen therapy

FIG. 4: Correlation between PaO₂ and SpO₂

FIG. 5: Correlation between FiO₂ and PAO₂

FIG. 6: Correlation between flowO₂ and FiO₂ in the flow range 0-20 liters/min

### DETAILED DESCRIPTION

The aspects and embodiments of the invention shown in the figures are exemplary of the invention and shall not be construed as limiting the invention thereby. In the figures, like numbers correspond to like elements.

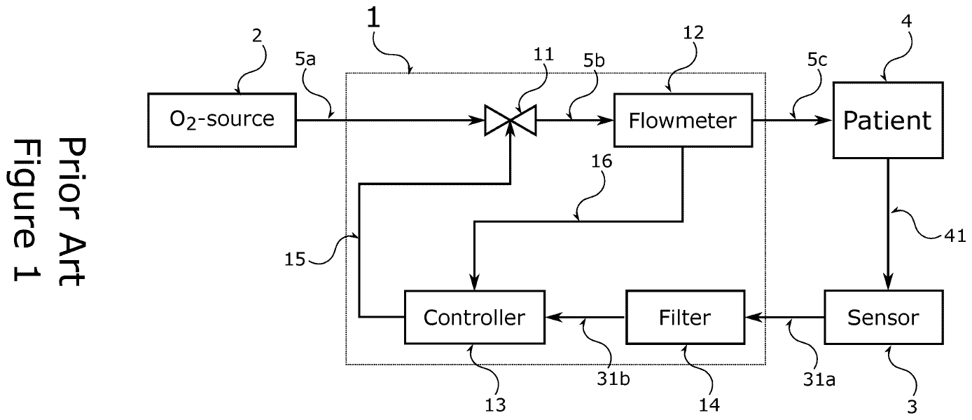

In FIG. 1 there is shown a standard double closed-loop regulated device (1) for automated oxygen therapy in accordance with the general instructions in WO 2006/110812. Such a standard double closed-loop regulated device (1) for regulating oxygen for automated oxygen therapy comprise an adjustable valve (11) for regulating a flow of oxygen, a flowmeter (12) for measuring a flow of oxygen, a controller (13) configured (15) for controlling a flow of oxygen through the adjustable valve (11) by adjusting the adjustable valve (11); the adjustable valve (11) and flowmeter (12) arranged consecutively for providing a flow path for oxygen (5a-c) through the device (1) such that, in use, oxygen from an oxygen source (2) can be provided to a point of use, such as being provided to a patient (4), via the device (1); wherein the flowmeter (12) is configured (16) for, in response to a flow of oxygen set by the controller (13), providing a first closed-loop feedback signal to the controller (13) comprising information on the measured flow of oxygen, for adjusting the adjustable valve (11) to provide a predetermined flow of oxygen; the controller (13) further configured (31a-b) for receiving a second closed-loop feedback signal from a sensor (3) configured (41) for measuring, in use of the device (1), a physiological data of a patient (4) receiving oxygen at the point of use, and providing the physiological data of the patient (4) to the controller as the second closed-loop feedback signal, optionally via a signal filter (14) conditioning the second closed-loop feedback signal; whereby the controller (13) in response to the second closed-loop signal comprising the physiological data of the patient (4) adjusts the predetermined flow of oxygen iteratively until the measured physiological data of the patient (4) corresponds to preset physiological data for the patient (4).

In the double closed-loop regulated device (1) for regulating oxygen for automated oxygen therapy detailed in WO 2006/110812 the presence of a signal filter (14) was considered necessary for conditioning the signal comprising the measured physiological data of the patient in order to obtain a true measured physiological data from the sensor, the sensor in WO 2006/110812 preferably being a pulse oximeter. In the double closed-loop regulated devices for regulating oxygen for automated oxygen therapy of the present invention, a signal filter (14) may or may not be present depending on the quality of the signal comprising the physiological data of a patient received from the sensor during automated oxygen therapy.

The present invention details improvements to the regulation of the oxygen flow via the adjustable valve (11) during heightened oxygen consumption by a patient receiving automated oxygen therapy by providing a control algorithm (100) whereby the controller (13) further is configured for, respectively arranged for, executing the control algorithm (100) in response to measured patient physiological data received from the sensor (3) indicating that the patient (4) being monitored is performing an activity involving physical exertion. The controller (13), in response thereto, will perform an iterative adaptation of the oxygen flow through the adjustable valve (11) using a predictive oxygen consumption model (200) for compensating for a heightened oxygen consumption during the physical exertion.

In accordance with the present invention, there is herein disclosed a double closed-loop regulated device (10) for regulating a flow of oxygen for automated oxygen therapy, the device (10) comprising an oxygen flow path (5a-c) for passing a flow of oxygen from a source of oxygen (2) via the device (10) for providing a controlled flow of oxygen, the device (10) comprising an adjustable valve (11) and a flowmeter (12) arranged consecutively on the oxygen flow path (5a-c), and a controller (13) configured (15) for controlling a provided flow of oxygen through the adjustable valve (11) by adjusting the adjustable valve (11) in response to feedback (16,31) from the flowmeter (12) and a sensor (3) for measuring physiological data comprising patient SpO₂ and pulse rate (PR), the sensor (3) configured for being operatively connected (31) to the controller (13); the controller (13) further configured for executing a control algorithm (100) in response to a change in measured physiological data received from the sensor (3) indicating a patient activity involving physical exertion; the control algorithm (100) permitting an iterative adaptation of the flow of oxygen through the adjustable valve (11) by using a predictive oxygen consumption model (200) for compensating for an increased patient oxygen consumption during physical exertion.

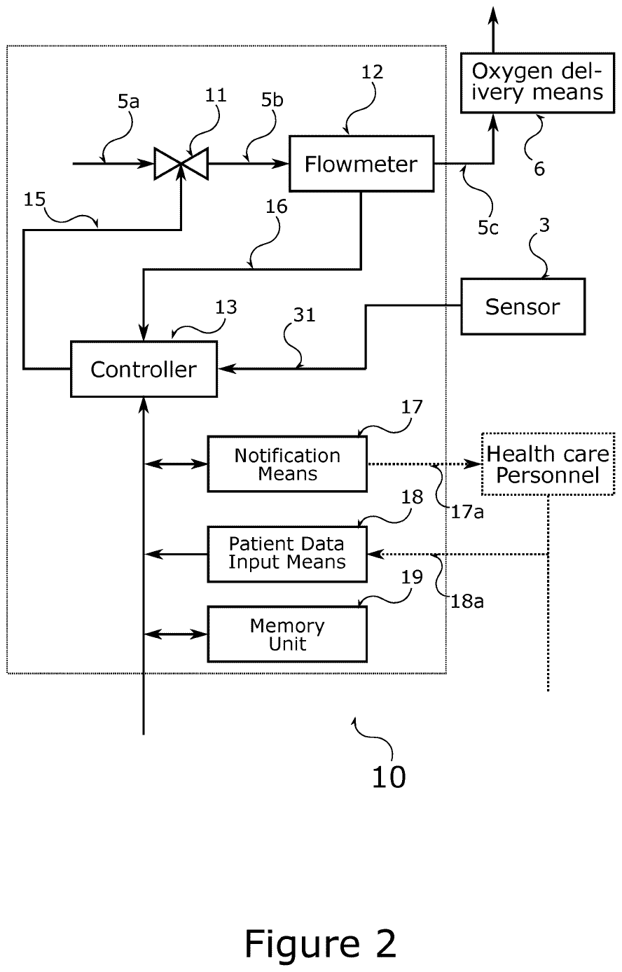

The device (10) of the invention and embodiments thereof are exemplarily illustrated in FIG. 2.

As is customary in the art, oxygen delivery means (6) are usually required for proper delivery of a flow of oxygen to a patient (4), such as oxygen masks, nasal oxygen cannulas etc., the oxygen delivery means (6) comprised in the oxygen flow path downstream (5c) of the flowmeter (12). Such oxygen delivery means (6) are in general known to the skilled person and are employed, in relation to the present invention, as known in the art. In some embodiments of the present device (10), oxygen delivery means (6) may be comprised in the device. However, normally the device (10) of the invention is configured for being operatively connected to such oxygen delivery means (6) as are known in the art, which thereby can be easily mounted and/or replaced for patient safety and hygiene.

In preferred embodiments of the method (20) and device (10) of the invention, the adjustable valve (11) comprises actuation means arranged for adjustment of the oxygen flow from an initial flow rate value to a next flow rate value, when receiving from the controller (13) a next flow rate value. In preferred embodiments thereof, the adjustable valve (11) is a PID-valve.

In preferred embodiments of the method (20) and device (10) of the invention, the sensor (3) is a pulse oximeter.

In preferred embodiments, the device (10) of the invention comprises a sensor (3) operatively connected (31) to the controller (13). In even more preferred embodiments thereof, the sensor (3) is a pulse oximeter.

In an embodiment, the device (10) of the invention comprises a signal filter (14) operatively arranged between (31a,31b) the sensor (3) connected (31) to the controller (13).

In an embodiment of the device (10) of the invention, the device (10) comprises a memory unit (19) operatively connected to the controller (13) for permitting provided patient data, such as e.g., patient treatment data or expected patient physiological data, to be stored and accessed by the controller (13).

In accordance with the invention, the controller (13) of the device of the invention is further configured for executing a control algorithm (100) in response to measured physiological data received from the sensor (3) indicating a patient activity involving physical exertion; the control algorithm (100) permitting an iterative adaptation of the flow of oxygen through the adjustable valve (11) using a predictive oxygen consumption model (200) for compensating for a heightened patient oxygen consumption during physical exertion.

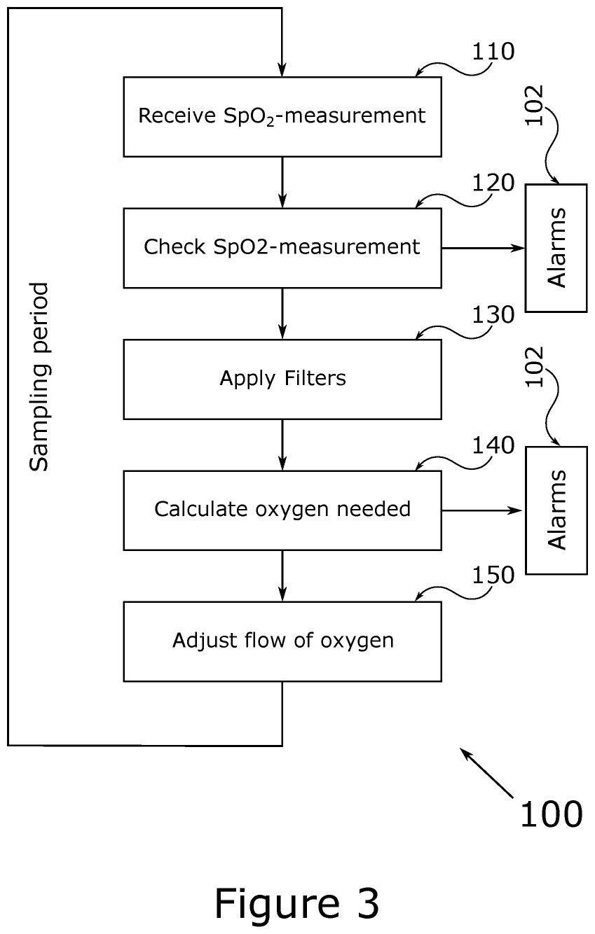

In accordance with the present invention, the control algorithm (100) can be implemented in accordance with the flow chart shown in FIG. 3, wherein the control algorithm (100) is shown in a preferred embodiment. In the preferred embodiment the control algorithm (100) comprises the steps of i. Receiving (110) a measured physiological data on the patient (4) from the sensor (3) comprising at least SpO₂ and pulse rate (PR); iv. Calculating (140), using a predictive oxygen consumption model (200), a predictive oxygen supply needed by the patient (4) and a predictive oxygen flow rate value; and v. Adjusting the oxygen flow rate through the adjustable valve (11) to the predictive oxygen flow rate. In an embodiment thereof, the predictive oxygen flow rate is provided to the adjustable valve (11) as a next flow rate value.

In embodiments thereof, the control algorithm (100) may optionally comprise between steps i. and iv., one or more of the steps of ii. Checking (120) if the received physiological data is valid physiological data and providing an error (102) if not; iii. Filtering (130) the received physiological data. In this context, the steps of ii. Checking (120) and iii. Filtering (130) rely on well-known techniques in the art, e.g., linear interpolation and low pass filters relying thereupon, and these are as such not further detailed herein, rather it is considered that the skilled person is capable of employing such checks and filters in accordance with the current state of the art.

As shown in FIG. 3, it is intended that the control algorithm (100) is repeated continuously at a sampling period. Preferably, the sampling period is sufficiently small to allow for the predictive adjustment to be physiologically relevant. However, as the patients receiving automated oxygen therapy are so ill that any adjustment is preferable over no adjustment, the sampling period is not a critical parameter. Rather, the inherent adjustment lag of the adjustable valve (11) will be the determinant of the meaningful adjustment rate possible. However, in order to receive an optimal adjustment of the oxygen flow, the sampling period should be kept low, e.g., not higher than any inherent adjustment lag of the adjustable valve (11). In the experiments and simulations presented herein below, the sampling period was shorter than the inherent adjustment lag of the adjustable valve (11).

The present invention builds on present inventor's clinical experience leading present inventor to the surprising realization that oxygen saturation in a patient, measured as SpO₂, and the fraction of O₂ in a patient's inspired air, measured as FiO₂, are correlated such in devices of the present art, that pairs of patient SpO₂ and FiO₂ can serve as a predictive oxygen consumption model (200) for compensating for a heightened patient oxygen consumption during physical exertion and for regulating the flow of oxygen during patient physical exertion.

In one embodiment, the flow of oxygen is regulated in accordance with the below Equation (1):

$$Δ Flow ∝ \frac{{SpO}_{2} ( target ) - {SpO}_{2} ( actual )}{{FiO}_{2} ( next ) - {FiO}_{2} ( actual )} = \frac{{SpO}_{2} ( T ) - {SpO}_{2} ( A )}{Δ {FiO}_{2} ( N - A )} \qquad ( 1 )$$

wherein the numerator term is adaptive to the measured values of oxygen saturation in a patient measured as SpO₂ with respect to a target SpO₂, and the denominator term is predictive by the term FiO₂ (next).

Equation (1) accordingly constitutes a predictive oxygen consumption model (200) which is suitable for use with the control algorithm (100) for permitting an iterative adaptation of the flow of oxygen through the adjustable valve (11) thereby compensating for a heightened patient oxygen consumption during physical exertion.

The desired target SpO₂ is a preset value having clinical relevance. In most clinically relevant scenarios, the target SpO₂ is typically set between 88% to 95% of maximum possible oxygen saturation in the patient with a safety interval of typically ±1% around the target SpO₂.

In general, while it is for the medical practitioner to determine for a given patient a desired target SpO₂ and/or safety interval as a preset value, in embodiments of the invention, the controller (13) is configured for receiving, preferably receiving as input patient data a desired target SpO₂ and a desired safety interval as a preset value. In some embodiments of the invention, the desired target SpO₂ and the desired safety interval are preset manufacturer's values.

Accordingly, in an embodiment of the control algorithm (100) iterative adaptation of the flow of oxygen through the adjustable valve (11) is performed in accordance with Equation (1) until a measure value is received from the sensor (3) for an actual SpO₂ falling within the desired safety interval around the desired target SpO₂.

While it is possible to directly measure the fraction of oxygen in the inspired air, it is more useful in the present invention to rely on tabulated values for the fraction of inspired oxygen, since the usefulness of a predictive oxygen consumption model (200), such as the above given Equation (1), relies on knowledge of the fraction of inspired oxygen for determining the flowrate change of oxygen from the device of the present invention.

TABLE 1
Relation between oxygen flow and changes in FiO₂

| Oxygenflow | | |
|---|---|---|
| [l/min] | FiO₂ | ΔFiO₂ |
| ≤1.0 | 0.250 | 0.040 |
| 2.0 | 0.290 | 0.040 |
| 3.0 | 0.330 | 0.040 |
| 4.0 | 0.365 | 0.035 |
| 5.0 | 0.400 | 0.035 |
| 6.0 | 0.430 | 0.030 |
| 7.0 | 0.460 | 0.030 |
| 8.0 | 0.485 | 0.025 |
| 9.0 | 0.510 | 0.025 |
| 10.0 | 0.530 | 0.020 |
| 11.0 | 0.550 | 0.020 |
| 12.0 | 0.565 | 0.015 |
| 13.0 | 0.580 | 0.015 |
| 14.0 | 0.590 | 0.010 |
| 15.0 | 0.600 | 0.010 |
| >15.0 | 0.600 | 0.010 |

When tabulated values for the fraction of inspired oxygen are used, Equation (1) rewrites as Equation (1a) below:

$$Δ Flow ∝ k \frac{{SpO}_{2} ( T ) - {SpO}_{2} ( A )}{Δ {FiO}_{2} ( N - A )} \qquad ( 1 a )$$

wherein the factor k is a device dependent calibration constant, which in the devices of the present invention come predetermined as a preset manufacturer's value.

Accordingly, in an embodiment of the control algorithm, the predictive oxygen consumption model (200) is given by Equation (1b).

$$Δ Flow = k \frac{{SpO}_{2} ( T ) - {SpO}_{2} ( A )}{Δ {FiO}_{2} ( N - A )} \qquad ( 1 b )$$

When the device (10) of the invention is in use and the patient (4) has the sensor equipped, the sensor (3) continuously measures the patient's SpO₂ and pulse rate (PR). When the patient is resting, the iterative double closed-loop regulation of the devices in the art and of the present device (10) assure an optimized oxygen supply to the patient. This again leads to stable patient SpO₂ and pulse rate during rest.

This changes when the patient begins to physically exerting him- or herself. Due to the severity of the general patient condition, SpO₂ rapidly drops, and the pulse rate increases, even at a level of physical exertion, which in healthy persons would appear minor, such as moving from one part of the patient's home to another part of the same home, e.g., from the living room to the kitchen (c.f. also the abovementioned clinical studies, wherein a 5-to-10-minutes' walk at slow pace was found to exhaust the study subjects).

However, given that patients receiving automated oxygen therapy are monitored using pulse-oximetry, detection of a physical exertion in a patient will be correlated directly to the rate of change (dropping) of SpO₂ or (increasing) pulse rate. This is used in the present device (1) and algorithms (100).

As such, in the context of the present disclosure, determination of if a patient is experiencing physical exertion is determined by the controller (13) in response to measured physiological data received from the sensor (3) carried by the patient receiving oxygen therapy.

It is therefore beneficial if the controller (13) is further arranged for executing the control algorithm (100) if a calculated resting average patient physiological data comprising at least one of SpO₂ and pulse rate, but preferably both, is subceeded (SpO₂—oxygen saturation drops), respectively superseded (PR—pulse rate goes up) relative to a calculated or preset resting patient value.

Accordingly, in an embodiment of the present invention, the controller (13) is arranged for calculating a calculated resting patient average physiological data comprising at least one of SpO₂ and pulse rate, but preferably both, by determining an average value for at least one of SpO₂ and pulse rate, but preferably both, when averaged over a preset averaging time.

In an embodiment of the present invention, the control algorithm (100) may comprise calculating a calculated resting average patient physiological data comprising at least one of SpO₂ and pulse rate, but preferably both, by determining an average value for at least one of SpO₂ and pulse rate, but preferably both, when averaged over a preset averaging time.

The preset averaging time can e.g., be 1 min, 2 min, 3 min, 4 min, or 5 min. The preset averaging time may be longer, but this is in general not necessary.

The preset rest value for the resting patient physiological data comprising SpO₂ and pulse rate will normally be set by a medical practitioner based on appropriate clinical standards and subsequently, in an embodiment of the present invention, provided to the device (10) of the invention as provided patient data.

It is however preferably to provide a calculated resting patient value for the resting patient physiological data comprising SpO₂ and pulse rate by setting a rest value safety margin on the calculated resting average patient physiological data, such as e.g., 10% of the calculated resting patient physiological data, 15%, 20%, 25%, or 30% of the calculated resting average patient physiological data, whereby the calculated resting patient value is continuously adapted to the calculated resting patient physiological data.

Thereby, in an embodiment of the invention, the controller (13) can be arranged for registering a change, preferably a change averaged over a preset time, in either or both of SpO₂ and/or pulse rate exceeding, respectively subceeding, the respective calculated resting average patient physiological data values, and permit execution of the control algorithm (100) in accordance with the present invention.

The rest value safety margin will normally be set by a medical practitioner and subsequently, in an embodiment of the present invention, provided to the device (10) of the invention as provided patient data, however in some embodiments, the rest value safety margin is a preset manufacturer's value.

While treatment using oxygen flow rates in excess of 5 liters per minute (l/min) per se has a low risk for unwanted side effects of treatment (contrary to too low oxygen flow), there are associated and unwanted side effects due to a too high oxygen flow, such as dryness in the mouth, which are preferably to avoid.

Since, during exertion oxygen requirements in all humans are increased (in particular, in vulnerable individuals comprised in the relevant patient groups for automated oxygen therapy), for this reason, during exertion, the controlled oxygen flowrate may be in excess of 15 l/min over a short time span, such as e.g., 10 minutes.

However, both healthy humans as well as persons suffering from e.g., COPD are at risk from hypercapnia if oxygen saturation in the blood stream prevents adequate release of carbon dioxide from the cells, such as can occur at very high oxygen flowrates.

Accordingly, in embodiments of the present invention, the controller (13) is further configured for limiting the controlled oxygen flowrate, if the controlled oxygen flowrate exceeds a preset maximum oxygen flowrate value, such as a preset maximum oxygen flowrate value of 10 l/min, 12.5 l/min, or 15 l/min for a time span longer than a preset maximum time span value of 15 min, 10 min, or 5 min.

In embodiments of the present invention, the controller (13) is further configured for notifying health care personnel, preferably notifying health care personnel using notification means (17) comprised in the device (10) of the invention, if the controlled oxygen flowrate exceeds a preset maximum oxygen flowrate value, such as a preset maximum oxygen flowrate value of 10 l/min, 12.5 l/min, or 15 l/min for a time span longer than a preset maximum time span value of 15 min, 10 min, or 5 min.

Further, the patient data, preferably the input patient data, may comprise a hazard level of average patient SpO₂ and/or a critical level of average patient SpO₂. Further, the patient data, preferably the input patient data, may comprise a preset maximum oxygen flowrate value and/or a preset maximum time span value.

In the art, the hazard level is often taken to be 94-97% of minimum average patient SpO₂, and/or the critical level is taken to be 80% of minimum average patient SpO₂. While it is for the medical practitioner to determine for a given patient critical and hazard levels of minimum average SpO₂ for a given patient, in embodiments of the invention, the controller (13) is configured for receiving, preferably receiving as input patient data, requisite hazard and/or critical levels of average patient SpO₂ during patient exertion, and in response to a received measured patient SpO₂ subceeding a requisite hazard and/or critical level of average patient SpO₂ during patient exertion increase the controlled flow of oxygen to a preset maximum flowrate of oxygen for a preset maximum time span. In an embodiment thereof, the controller (13) is arranged to resume iteratively regulating the flowrate of oxygen through the adjustable valve (11), after the preset maximum timespan.

In an embodiment of the double closed-loop regulated device (10) for regulating a flow of oxygen for automated oxygen therapy, the controller (13) is configured for returning an error (102) of process if a criterion for error (102) of process is passed.

In an embodiment of the method (20) and device (10), the controller (13) is configured for returning an error (102) of process during execution of the method (20) of the invention, if a criterion for error is passed. Exemplary, but non-limiting, embodiments detailing criteria for error (102) of process are detailed herein below. In a preferred embodiment of the device (10), the controller is configured for returning an error (102) of process, the controller (13) is further configured for providing information of a returned error (102) of process to notification means (17) comprised in the device (10) of the invention. By having the controller (13) configured for returning information on errors in the executed processes of the invention to notification means, safety in outpatient treatment is enhanced.

Accordingly, in one embodiment of the device (10) of the invention, the device (10) comprises notification means (17) operatively connected to the controller (13) and configured for providing a notification in the event of an error (102) of process.

In one embodiment of the notification means (17) configured for providing a notification in the event of an error (102) of process, the notification means (17) is configured (17a) for contacting authorized health care personnel. Such notification can e.g., comprise an automated email or call notification to a ward. In general, the construction and implementation of notification means (17) for health care and in health care devices are considered within the skills of the person in the art and outside the scope of the present invention.

In an embodiment of the device (10) of the invention, the device (10) comprises patient data input means (18) operatively connected to the controller (13) for permitting (18a) authorized health care personnel to provide patient data, such as e.g., patient treatment data or expected patient physiological data, to the controller (13).

In embodiments, the controller is set to adjust the flow rate of oxygen to either the maximum long-term or short-term flow rate, respectively, if the average patient SpO₂ during physical exertion subceeded the aforementioned hazard or critical average patient SpO₂-levels.

Accordingly, in one embodiment, the controller (13) is configured to return an error (102) of process if patient physiological data is not received from the aforementioned sensor (3).

In embodiments, where health care personnel are required to provide input patient data for treatment patient data, it is preferable to provide patient data input means (18) comprising selection means, preferably comprising selection means comprising a selection menu, comprising preset patient treatment input patient data for selecting by health care personnel as input patient data for treatment patient data. Thereby risks of accidentally setting patient treatment data outside suitable clinical limits can be minimized. The design and operation of such selection means comprised in patient data input means (18) are well-known in the art and outside the scope of the present invention.

Accordingly, in an embodiment of the control algorithm (100), the predictive oxygenation model (200) is given as in Equation (1c) below:

$$Δ Flow ∝ k \frac{{SpO}_{2} ( T ) - {SpO}_{2} ( A )}{Δ {FiO}_{2} ( N - A )} \cdot FR ( PR ) \qquad ( 1 c )$$

Herein the term FR(PR) is a flowrate response factor (FR) adaptive to a change in the measured pulse rate (PR). The term FR(PR) serves to accelerate the flowrate change during the initial stages of a patient physical exertion, where the pulse rate of the patient exercising physically increases rapidly from the resting pulse rate towards a (more) stable work pulse rate. The exact nature of the underlying function governing the flowrate response to the change in pulse rate is less important as long as it secures a rapid initial response and convergence towards a flowrate response factor of 1 when a stable work pulse rate is registered by measurement using the sensor (3). At the later point, flowrate change in accordance with Equation (1) is adequate. An example of a function providing a desired flowrate response factor could be e.g., an exponential function dependent on ΔPR.

In an embodiment of the control algorithm (100), the predictive oxygenation model (200) is given as in Equation (1d) below:

$$Δ Flow = k \frac{{SpO}_{2} ( T ) - {SpO}_{2} ( A )}{Δ {FiO}_{2} ( N - A )} \cdot FR ( PR ) \qquad ( 1 d )$$

In an embodiment of the control algorithm (100), the predictive oxygenation model (200) is given as in Equation (1e) below:

$$Δ Flow ∝ k \frac{{SpO}_{2} ( T ) - {SpO}_{2} ( A )}{Δ {FiO}_{2} ( N - A )} \cdot ( 1 - \frac{{PAO}_{2} - {PaO}_{2}}{{PAO}_{2}} ) \qquad ( 1 e )$$

The term (PAO₂-PaO₂) represents the alveolar-arterial oxygen-difference, which is a measure of the pulmonary condition. The higher the value, the worse the condition of the patient performing physical exertion. The term cannot (chemically and biologically) subceed zero but may subceed zero in the herein presented models under patient use conditions. If the term in a model calculation subceeds zero, it is reset to a value of 1 in the control algorithm (100) of the present invention and the flow response recalculated as in Equation (1e) under the new conditions.

It is herein included into the predictive oxygenation model (200) as it permits the flow response to be accelerated individually for a given patient receiving automated oxygen therapy based on the same patients measured oxygen consumption ability at a given flowrate of oxygen provided from the device (10) of the invention and measured as SpO₂ by the sensor (3).

Firstly, a relation between PaO₂ and SpO₂ can be derived from the Severinghaus formula (as mmHg):

$${SpO}_{2} = {( 2 \text{.34} \times {10}^{3} {( {( {PaO}_{2} )}^{3} + 150 {PaO}_{2} )}^{- 1} + 1 )}^{- 1}$$

but is preferably tabulated for use with the present invention in accordance with the below Table 2:

TABLE 2
Relation between SpO₂ and PaO₂ derived
from the Severinghaus formula:

| SpO₂ | PaO₂ |
|---|---|
| ≤70% | 4.88 |
| 71% | 4.97 |
| 72% | 5.06 |
| 73% | 5.15 |
| 74% | 5.24 |
| 75% | 5.34 |
| 76% | 5.44 |
| 77% | 5.55 |
| 78% | 5.66 |
| 79% | 5.78 |
| 80% | 5.91 |
| 81% | 6.04 |
| 82% | 6.18 |
| 83% | 6.33 |
| 84% | 6.50 |
| 85% | 6.66 |
| 86% | 6.85 |
| 87% | 7.07 |
| 88% | 7.30 |
| 89% | 7.55 |
| 90% | 7.82 |
| 91% | 8.15 |
| 92% | 8.52 |
| 93% | 8.95 |
| 94% | 9.45 |
| 95% | 10.1 |
| 96% | 10.9 |
| 97% | 12.1 |
| 98% | 13.8 |
| ≥99% | 17.5 |

Likewise, the relation between FiO₂ and PAO₂ is given by the alveolar gas equation:

$${PAO}_{2} = ( {P}_{ATM} - {P}_{H 2 O} ) - ( {PaCO}_{2} / RER ) \cdot {FiO}_{2}$$

Which under normal conditions body conditions at sea level, normal PaCO₂ and normal metabolism (RER) equates to:

$${PAO}_{2} = ( 101.33 kPa - 6.27 kPa ) - ( \frac{5.3 kPa}{0.80} ) \cdot {FiO}_{2} = 95.06 kPa - ( \frac{5.3 kPa}{0.80} ) \cdot {FiO}_{2}$$

wherein FiO₂ is as given for a specific flowrate by Table 1.

The term

$$( 1 - \frac{{PAO}_{2} - {PaO}_{2}}{P A {O}_{2}} )$$

in Formula (1e) therefore serves to dampen the response of the flowrate change for patients with severe disease conditions, where a rapid flowrate change may be ill received by the patients lungs where these have insufficient capacity for transporting air from the alveoli and into the arterial bloodstream.

Accordingly, in an embodiment of the control algorithm (100), the predictive oxygenation model (200) is given as in Equation (1f) below:

$$Δ Flow = k \frac{{SpO}_{2} ( T ) - {SpO}_{2} ( A )}{Δ {FiO}_{2} ( N - A )} \cdot ( 1 - \frac{{PAO}_{2} - {PaO}_{2}}{{PAO}_{2}} ) \qquad ( 1 f )$$

In a further embodiment of the control algorithm (100), the predictive oxygenation model (200) is given as in Equation (1g) below, combining flow regulation with the terms for accelerating and dampening the regulation of the flow in response to a patient's physical exercise.

$$Δ Flow = k \frac{{SpO}_{2} ( T ) - {SpO}_{2} ( A )}{Δ {FiO}_{2} ( N - A )} \cdot ( 1 - \frac{{PAO}_{2} - {PaO}_{2}}{{PAO}_{2}} ) \cdot FR ( PR ) \qquad ( 1 g )$$

In accordance with the invention, there is further detailed herein the use of a double closed-loop regulated device (10) for regulating a flow of oxygen for automated oxygen therapy according to any of the herein detailed embodiments for use in a method for providing automated oxygen therapy to a patient (4) according to any of the herein detailed methods.

In accordance with the invention, there is further detailed herein a double closed-loop regulated device (10) for regulating a flow of oxygen for automated oxygen therapy according to any of the herein detailed embodiments for use in a method for providing automated oxygen therapy to a patient (4) according to any of the herein detailed methods.

In the calculations above, the needed change in oxygen flow (ΔflowO₂) from the deviation of actual oxygen saturation from target saturation (ΔspO₂) in a closed-loop system is done essentially by linear approximation, which is satisfactory at the initiation of physical exertion. However, while it is possible to estimate the needed change in oxygen flow (ΔflowO₂) from the deviation of actual oxygen saturation from target saturation (ΔSpO₂) in a closed-loop system by linear approximation as done above, the accuracy of the approximation is rapidly lost, if the physical exertion by the patient is carried out for a period of time long enough for a new stable oxygen consumption level to be reached.

In a fully modelled version of the predictive oxygen consumption model (200) a number of consecutive linear and non-linear correlations to be considered in this estimation, and thus it is not possible to simply estimate that:

$$Δ {flow O}_{2} = constant * Δ {SpO}_{2}$$

Rather, in considering all the relevant correlations, a oxygen molecule must be followed from the oxygen delivering device, such as a nasal cannula, into the central airways, further down into the alveoli in the lungs, from the alveoli to the arterial bloodstream, and from there to the binding of oxygen to the hemoglobin molecule.

In the below model, the present inventor has carefully considered each step must whereby the relevant oxygen parameters can be calculated in an improved manner:

In the below calculations wherein, in accordance with the above definitions, oxygen flow in the oxygen delivering device is flowO₂, fraction of oxygen in the inspired air in the central airways is FiO₂, partial pressure of oxygen in the alveoli is P_{A}O₂, partial pressure of oxygen in arterial blood is PaO₂, and saturation of the hemoglobin molecules with oxygen is SpO₂.

As discussed above, in the closed-loop system only know the delivered flowO₂ and the measured SpO₂ are determinable, hence from change in SpO₂ it is necessary to estimate the changes in FiO₂, P_{A}O₂ and PaO₂ in order to estimate how much to change the flow of oxygen through the adjustable valve (11).

In the present model below, the present inventor has found that an improved predictive oxygen flow regulation can be achieved through application of the below four linearization correlations (f1-f4), wherein:

$$Δ {SpO}_{2} = {f}_{1} * Δ {PaO}_{2}$$

$$Δ {PaO}_{2} = {f}_{2} * Δ {P}_{A} {O}_{2}$$

$$Δ {P}_{A} {O}_{2} = {f}_{3} * Δ {FiO}_{2}$$

$$Δ Fi {O}_{2} = {f}_{4} * Δ {flow O}_{2}$$

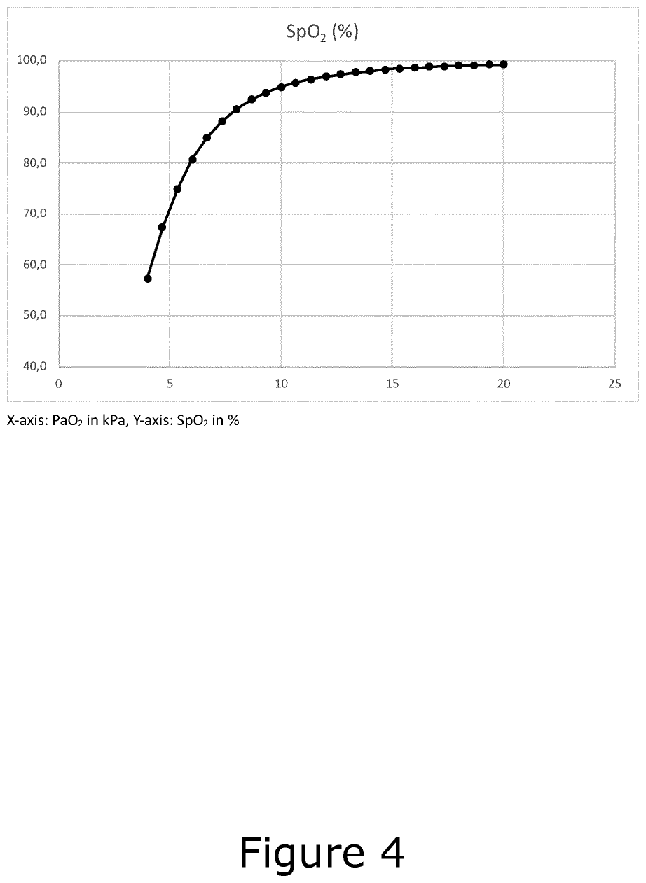

The four functions (f₁-f₄) are as follows:
f₁ is Severinghaus' formula, with PaO₂ entered in mmHg, cf. FIG. 4:

$${f}_{1} : {SpO}_{2} = {( 23 , 400 * {( {PaO}_{2}^{3} + 150 * {PaO}_{2} )}^{- 1} + 1 )}^{- 1}$$

f₂ is the alveoli-arterial gradient for a given patient:
f₂: P_{A}O₂—PaO₂=A-a-gradient (alveoli-arterial gradient)

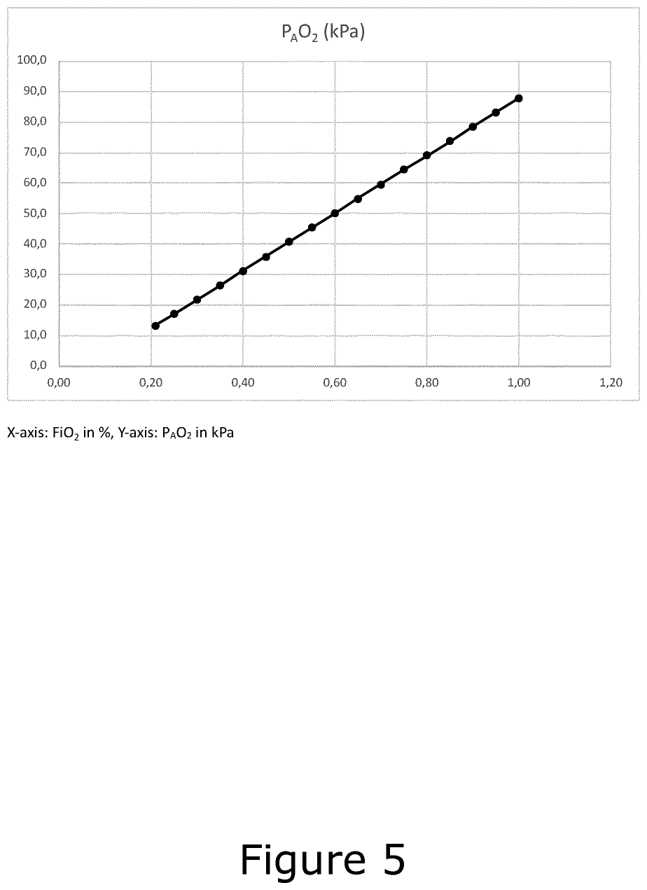

The A-a-gradient increases with increasing disease severity. The gradient can be determined by iteration titration with oxygen, as a small change in SpO₂ in response to a given flow change indicates a large A-a-gradient. As the present device (10) are suitable for iteratively titrating the oxygen flow, in one embodiment the controller (13) is further arranged for performing a determination of the A-a-gradient for a given patient receiving automated oxygen treatment using a device (10) of the present invention.
f₃ is the alveolar gas equation, cf. FIG. 5:

$${f}_{3} : {P}_{A} O 2 = {FiO}_{2} ( {P}_{A T M} - {P}_{H 2 O} ) - {PaCO}_{2} / RER$$

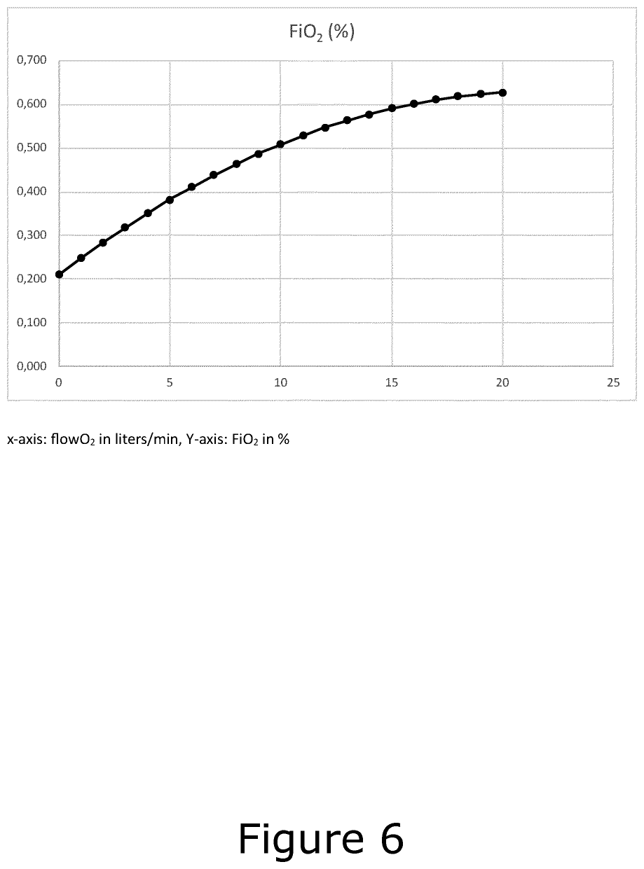

Under normal circumstances the following values can be estimated: P_{ATM}=101 kPa, P_{H2O}=6.3 kPa, PaCO₂=5.4 kPa, RER=0.8 (FIG. 5), and:
f₄ is a device dependent FiO₂-response to oxygen flow (cf. FIG. 6):

The linearization function f₄ must be individually determined for a given device for automated oxygen therapy. In the present Applicant's O2matic devices, the response equation is well fitted using a 2^{nd} degree polynomial function as given below:

$${f}_{4} : {FiO}_{2} = {c}_{1} * {( {flow O}_{2} )}^{2} + {c}_{2} * ( {flow O}_{2} ) + {c}_{3}$$

In FIG. 6, an estimation of best fit in the flow range 0-20 liters/min, (FIG. 6), for a device in use is presented, wherein the function f4 is well approximated as:

$${f}_{4} : {FiO}_{2} = - 0 \text{.0009} * {( {flow O}_{2} )}^{2} + 0 \text{.0389} * ( {flow O}_{2} ) + 0 , 21$$

It follows from the above that the change in the flow of oxygen can be calculated from the measured change in the SpO₂ by consecutive calculation of a set of at least four linearized correlations, such that ultimately, the measured change in SpO₂ correlates to a predicted change in the flow of oxygen as:

$$Δ flow ( {O}_{2} ) = \frac{1}{{f}_{1} * {f}_{2} * {f}_{3} * {f}_{4}} * Δ {SpO}_{2} \qquad ( 2 )$$

wherein f₁-f₄ are as given above.

It necessarily follows that for Equation (2) to be useful as a predictive oxygen consumption model, the functions f₂ and f₄ must be supplied to the controller prior to operation of the device (10) of the invention. As oxygen titration for determining the alveoli-arterial gradient for a given patient and device calibration requires trained medical personnel, determination of f₂ and f₄ is outside the scope of the present invention. Rather, the functions f₂ and f₄ are in preferred embodiments supplied to the controller as input patient data prior to executing the control algorithm (100).

Accordingly, in a preferred embodiment of the present invention, the predictive oxygen consumption model (200) comprises calculating a change in the flow of oxygen through the adjustable valve (11) using a linearized correlation function correlating a change in the flow of oxygen through the adjustable valve (11) with a measured change in SpO₂.

In a more preferred embodiment of the present invention, the linearized correlation function is a linearized correlation function according to equation (2).

### CLOSING COMMENTS

The term “comprising” as used in the claims does not exclude other elements or steps. The term “a” or “an” as used in the claims does not exclude a plurality. A single processor or other unit may fulfill the functions of several means recited in the claims. Although the present invention has been described in detail for purpose of illustration, it is understood that such detail is solely for that purpose, and variations can be made therein by those skilled in the art without departing from the scope of the invention.

1. A double closed-loop regulated device for regulating a flow of oxygen for automated oxygen therapy, the device comprising an oxygen flow path for passing a flow of oxygen from a source of oxygen via the device for providing a controlled flow of oxygen, the device comprising an adjustable valve and a flowmeter arranged consecutively on the oxygen flow path, and a controller configured for controlling a provided flow of oxygen through the adjustable valve by adjusting the adjustable valve in response to feedback from the flowmeter and a sensor for measuring physiological data comprising patient SpO₂ and pulse rate, the sensor configured for being operatively connected to the controller; the controller further configured for executing a control algorithm in response to a change in measured physiological data received from the sensor indicating a patient activity involving physical exertion; the control algorithm arranged for permitting an iterative adaptation of the flow of oxygen through the adjustable valve by using a predictive oxygen consumption model for compensating for an increased patient oxygen consumption during physical exertion.

2. A double closed-loop regulated device for regulating a flow of oxygen for automated oxygen therapy according to claim 1, the device further comprising the sensor operatively connected to the controller.

3. A double closed-loop regulated device for regulating a flow of oxygen for automated oxygen therapy according to claim 1, wherein the sensor is a pulse oximeter.

4. A double closed-loop regulated device for regulating a flow of oxygen for automated oxygen therapy according to claim 1, wherein the device is configured for being operatively connected to oxygen delivery means, said oxygen delivery means for being comprised in the oxygen flow path downstream of the flowmeter.

5. A double closed-loop regulated device for regulating a flow of oxygen for automated oxygen therapy according to claim 4, wherein said oxygen delivery means are comprised in the oxygen flow path downstream of the flowmeter.

6. A double closed-loop regulated device for regulating a flow of oxygen for automated oxygen therapy according to claim 1, wherein the controller is configured for returning an error of process if a criterion for error of process is passed.

7. A double closed-loop regulated device for regulating a flow of oxygen for automated oxygen therapy according to claim 1, wherein the device further comprises notification means operatively connected to the controller and configured for providing a notification if a criterion for error of process is passed.

8. A double closed-loop regulated device for regulating a flow of oxygen for automated oxygen therapy according to claim 1, wherein the device further comprises patient data input means operatively connected to the controller for permitting at least patient data to be provided to the controller for configuring the controller for executing said control algorithm.

9. A double closed-loop regulated device for regulating a flow of oxygen for automated oxygen therapy according to claim 1, wherein the device further comprises a memory unit operatively connected to the controller for permitting at least provided patient data to be stored and accessed by the controller.

10. A double closed-loop regulated device for regulating a flow of oxygen for automated oxygen therapy according to claim 8, wherein the patient data comprises patient physiological data comprising patient SpO₂ and pulse rate.

11. A double closed-loop regulated device for regulating a flow of oxygen for automated oxygen therapy according to claim 1, wherein the predictive oxygen consumption model is given by Equation (1b):
$$Δ Flow = k \frac{{SpO}_{2} ( T ) - {SpO}_{2} ( A )}{Δ {FiO}_{2} ( N - A )} \qquad ( 1 b )$$

12. A double closed-loop regulated device for regulating a flow of oxygen for automated oxygen therapy according to claim 1, wherein the predictive oxygen consumption model comprises calculating a change in the flow of oxygen through the adjustable valve using a linearized correlation function correlating a change in the flow of oxygen through the adjustable valve (11) with a measured change in SpO₂.

13. A closed-loop regulated double device for regulating a flow of oxygen for automated oxygen therapy according to claim 1, where in the predictive oxygen consumption model, the linearized correlation function is a linearized correlation function given by Equation (2):
$$Δ flow ( {O}_{2} ) = \frac{1}{{f}_{1} * {f}_{2} * {f}_{3} * {f}_{4}} * Δ {SpO}_{2} \qquad ( 2 )$$
wherein f₁ is Severinghaus' formula,
f₂ is the alveoli-arterial gradient for a given patient,
f₃ is the alveolar gas equation, and
f₄ is a device dependent FiO₂-response to oxygen flow; and
wherein f₂ and f₄ are provided as input patient data prior to executing said control algorithm.
